(19) United States
(12) Patent Application Publication
(10) Pub. No.: US 2024/0392222 A1
(43) Pub. Date: Nov. 28, 2024

(54) Modular Solid Nano-Bioreactor

(71) Applicant: Government of the United States as represented by the Secretary of the Air Force, Wright-Patterson AFB, OH (US)

(72) Inventor: Oscar N. Ruiz, Bellbrook, OH (US)

(21) Appl. No.: 18/637,872

(22) Filed: Apr. 17, 2024

Related U.S. Application Data
(60) Provisional application No. 63/468,384, filed on May 23, 2023.

(51) Int. Cl. C12M 1/16; C12M 1/00; C12M 1/12

(52) U.S. Cl. CPC C12M 21/16; C12M 23/34; C12M 25/14; C12M 29/04

(57) ABSTRACT

The present invention relates to modular solid nano-bioreactor and method of using same. The present invention relates to modular solid nano-bioreactor and method of using same. Such modular solid nano-bioreactor are easily scalable, allow densely packed biofilms to be produced and to be immobilized in the modular solid nano-bioreactor which in turn permits easy purification, continuous fermentation and prevents the release of genetically modified organisms. Furthermore, the aforementioned biofilms protect the organisms from the fermentation process. Finally, the cell packing efficiency of the aforementioned modular solid nano-bioreactors leads to high cell density and high production levels.

### CROSS-REFERENCE TO RELATED APPLICATIONS

The present application claims priority to U.S. Provisional Application Ser. No. 63/468,384 filed May 23, 2023, the contents of such provisional application hereby being incorporated by reference in its entry.

### RIGHTS OF THE GOVERNMENT

The invention described herein may be manufactured and used by or for the Government of the United States for all governmental purposes without the payment of any royalty.

### FIELD OF THE INVENTION

The present invention relates to modular solid nano-bioreactors and method of using same.

### BACKGROUND OF THE INVENTION

For over 100 years, fermentation has been carried in batch bioreactors relaying on a liquid nutrient media to grow the bacterial or fungal inoculum. The cell density in liquid is highly dependent on biotic and abiotic factors such as oxygen, agitation, pH, and nutrient availability which have to be precisely controlled especially in large-scale bioreactors. Even with the best conditions possible, the fluid condition of the liquid does not allow for cells in constant movement to fill every available space in the liquid culture medium, meaning that cell density and hence limits product titers and total production. Another type of bioreactor in use today in experimental and some industrial applications are flow reactors. While flow bioreactors allow for continuous production of biomolecules, the growth rate and cell density achieved after stead-state is often much lower than that achieved on batch bioreactor. Finally, the accumulation of toxic byproducts and contamination in the liquid cultures of batch and flow bioreactors can highly affect the vigor of the microbial culture exposed to that environment.

Graphene oxide modular solid bioreactors trigger the formation of biofilms having a thick extracellular polymeric substance and biofilms having multiple layers of bacteria arranged in thick sheets. These characteristics present the possibility of modulating the biofilm to increase the number of cells per surface area while enhancing the formation of extracellular factors leading to increased robustness. The nano-bioreactor potential for high titer production of biomolecules is expected to directly depend on the thickness of the biofilm, the density of the cells in the biofilm and the robust health of the cells. Biofilms are extremely resistance to biotic and abiotic factors, which makes them uniquely attractive for biofermentation processes in austere environments with sub-optimal growth conditions.

The semi-solid and solid GO/bio-film materials are expected to have considerable advantage over liquid fermentation systems in that they will facilitate development of continuous flow fermentation processes by trickling nutrients through the nano-bioreactor bed while providing easy purification of the secreted bioproduct as the cells are irreversibly immobilized into the nanomaterial and cannot escape. Also, the GO materials enhance the formation of thick biofilms that are expected to drastically increase the biomass and robustness of the culture for the biomanufacturing process, maximizing product output. The nano-biomaterial bed matrices will facilitate scalability and development of ruggedized modular reactors for agile biomanufacturing under distributed and austere conditions including space as they require minimal amount of liquids. The scalability of these systems also allow to go from small scale 1-100,000 L scales. Finally, because the cells are immobilized in the bioreactor, this drastically minimize the potential of inadvertently releasing genetically modified microorganisms to the environment.

### SUMMARY OF THE INVENTION

The present invention relates to modular solid nano-bioreactor and method of using same. Such modular solid nano-bioreactor are easily scalable, allow densely packed biofilms to be produced and to be immobilized in the modular solid nano-bioreactor which in turn permits easy purification, continuous fermentation and prevents the release of genetically modified organisms. Furthermore, the aforementioned biofilms protect the organisms from the fermentation process. Finally, the cell packing efficiency of the aforementioned modular solid nano-bioreactors leads to high cell density and high production levels.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 depicts a cross-sectional view of an embodiment of a modular solid nano-bioreactor with additional compression spacers to reduce pressure loss, having housing inlet (1), first compression spacer (2), first plurality of scaffolds having graphene oxide flakes disposed on said scaffolds (3), second compression spacer (4), second plurality of scaffolds having graphene oxide flakes disposed on said scaffolds (5), optional bed of graphene oxide fakes (6), second compression space (7), housing outlet (8), housing (9), cell growing on or embedded in the graphene oxide matrix (10).

FIG. 2 depicts a gel matrix comprised of graphene oxide nanomaterials and embedded cells capable of producing valuable products. Graphene Oxide gel matrix with embedded microorganisms (1), graphene\oxide component of the gel matrix (2), microbial cells in the gel matrix (3).

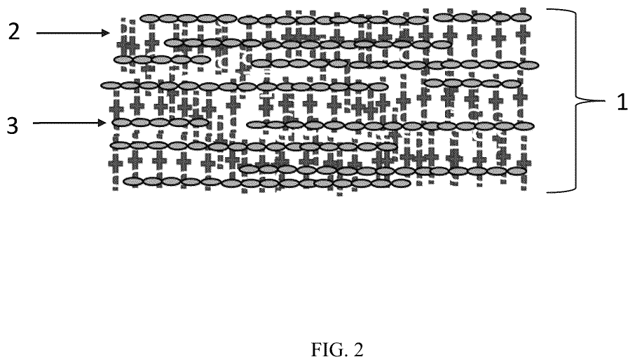

### DETAILED DESCRIPTION OF THE INVENTION

### Definitions

As used herein, the term “seeded” means inoculated prior to the start of the of the production run used to grow the desired microbes. A liquid media that contains the microorganism that is to be seeded may be run through the modular solid nano-bioreactor to seed the microorganism in the modular solid nano-bioreactor graphene oxide. Such liquid media may be a liquid nutrient media.

Unless specifically stated otherwise, as used herein, the terms “a”, “an” and “the” mean “at least one”.

As used herein, the terms “include”, “includes” and “including” are meant to be non-limiting.

As used herein, the words “about,” “approximately,” or the like, when accompanying a numerical value, are to be construed as indicating a deviation as would be appreciated by one of ordinary skill in the art to operate satisfactorily for an intended purpose.

As used herein, the words “and/or” means, when referring to embodiments (for example an embodiment having elements A and/or B) that the embodiment may have element A alone, element B alone, or elements A and B taken together.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Modular Solid Nano-Bioreactor

For purposes of this specification, headings are not considered paragraphs. In this paragraph, Applicants disclose a modular solid nano-bioreactor comprising walls that form a three dimensional housing, said housing comprising an interior chamber, at least one inlet passing through at least one of said walls to said interior chamber and at least one outlet passing through at least one of said walls to said interior chamber, said housing's chamber comprising a three dimensional structure comprising graphene oxide seeded with a microorganism and/or a cell, said microorganism and/or cell being embedded in and/or attached to the surface of said graphene oxide.

Applicants disclose the modular solid nano-bioreactor according to the previous paragraph wherein said graphene oxide is selected from Graphene oxide flakes, graphene oxide powder, graphene oxide hydrogels and/or solvgels, graphene oxide liquid solution, preferably graphene oxide flakes, graphene oxide powder, graphene oxide hydrogels and/or solvgels, more preferably graphene oxide flakes, graphene oxide hydrogels and/or solvgels, solution, most preferably graphene oxide flakes, and/or graphene oxide hydrogels

Applicants disclose the modular solid nano-bioreactor according to the previous two paragraphs wherein said microorganism is selected from the group consisting of bacteria, fungi, protist, archaea and mixtures thereof; and/or said cell is selected from the group consisting of eukaryotic cells, prokaryotic cells and mixtures thereof.

Applicants disclose the modular solid nano-bioreactor according to the previous paragraph wherein: said bacteria said bacteria are selected from the group consisting of gram negative bacteria, gram positive bacteria and mixtures thereof; preferably said gram negative bacteria are selected from the group consisting of Synechococcus, Synechocystis, Pseudomonas aeruginosa, Pseudomonas stutzeri, Pseudomonas frederiksbergensis, Pseudomonas putida, Pseudomonas, Acinetobacter venetianus, Acinetobacter calcoaceticus, Marinobacter hydrocarbonoclasticus, Marinobacter alkaliphilus, Marinobacter maritimus, Marinobacter squalenivorans, Rhodovulum imhoffii, Rhodovulum sulfidophilum, Achromobacter spanius, Achromobacter xylosoxidans, Achromobacter denitrificans, Desulfovibrio alaskensis, Desulfovibrio desulfuricans, Escherichia coli, Synechocystis sp., Synechococcus elongatus, and mixtures thereof, preferably said gram positive bacteria are selected from the group consisting of Gordonia sihwensis, Nocardioides luteus, Bacillus lincheniformis, Dietzia psychralcaliphila and mixtures thereof; said fungi are selected from the group consisting of filamentous fungi, yeasts and mixtures thereof; preferably said filamentous fungi are selected from the group consisting of Hormoconis resinae, Fusarium fujikuroi, Byssochlamys nivea, Apergillus versicolor, Byssochlamys sp BYSS01, Lecanicillium sp Lec01, and mixtures thereof, preferably said yeasts are selected from the group consisting of Saccharomyces cerevisiae, Schizosaccharomyces pombe, Yarrowia lipolytica, Meyerozyma guilliermondii, Candida tropicalis, Candida albicans, Debaryomyces, Rhodoturula mucilaginosa, Rhodoturula toruloides and mixtures thereof; said archaea are selected from the group consisting of methanobacterium formicicum, Methanobrevibacter smittii, Methanobrevibacter ruminantium, Halobacterium halobium, Halobacterium salinarum, Methanococcus voltae, and mixtures thereof; said protist are selected from the group consisting of algae, amoeba, euglena and mixtures thereof, preferably said algae are selected from the group consisting of Clamydomonas, volvox, Spirogyra, chlorella and mixtures thereof; and said eukaryotic cells are selected from the group consisting of mammalian cells, insect cells, plant cells and mixtures thereof, preferably said mammalian cells are human cells, mouse cells, monkey cells and mixtures thereof.

Applicants disclose the modular solid nano-bioreactor according to the previous four paragraphs wherein: said three dimensional structure comprising graphene oxide is selected from the group consisting of a three dimensional structure comprising porous graphene oxide, said three dimensional structure comprising porous graphene oxide not comprising a support scaffold; and/or a three dimensional structure comprising graphene oxide, said three dimensional structure comprising graphene oxide comprising a support scaffold comprising an exterior surface and optionally pores having walls said pores passing through said support scaffold, said graphene oxide being attached to at least a portion of said three dimensional structure comprising graphene oxide.

Applicants disclose the modular solid nano-bioreactor according to the previous four paragraphs wherein said three dimensional structure comprising graphene oxide is selected from the group consisting of a three dimensional structure comprising a graphene oxide cartridge, graphene oxide gel and/or graphene oxide mesh; and/or a three dimensional structure comprising graphene oxide, said three dimensional structure comprising graphene oxide comprising a support scaffold comprising an exterior surface and optionally pores having walls said pores passing through said support scaffold, said graphene oxide being attached to at least a portion of said three dimensional structure comprising graphene oxide, said support scaffold comprising pores having walls said pores passing through said support scaffold.

Applicants disclose the modular solid nano-bioreactor according to the previous four paragraphs wherein said three dimensional structure comprising graphene oxide is selected from the group consisting of a graphene oxide cartridge, and/or graphene oxide mesh; and/or a three dimensional structure comprising graphene oxide, said three dimensional structure comprising graphene oxide comprising a support scaffold comprising an exterior surface and optionally pores having walls said pores passing through said support scaffold, said graphene oxide being attached to at least a portion of said three dimensional structure comprising graphene oxide, said support scaffold comprising pores having walls said pores passing through said support scaffold, said graphene oxide being attached to the walls of said pores and said support scaffold's exterior surface.

Applicants disclose the modular solid nano-bioreactor according to the previous paragraph wherein said support scaffold comprises a graphene oxide mesh, a bead, a fiber, a tube, a nanotube, and/or nanoparticle all said bead, a fiber, a tube, a nanotube, and nanoparticle having a graphene oxide attached thereto.

Applicants disclose the modular solid nano-bioreactor according to the previous eight paragraphs, said modular solid nano-bioreactor comprising a compression spacer located between said inlet and said three dimensional structure comprising graphene oxide or between said outlet and said three dimensional structure comprising graphene oxide.

Applicants disclose the modular solid nano-bioreactor according to the previous paragraph wherein said compression spacer is located between said inlet and said three dimensional structure comprising graphene oxide.

Applicants disclose the modular solid nano-bioreactor according to the first eight paragraphs of this section titled “Modular Solid Nano-Bioreactor”, wherein said modular solid nano-bioreactor comprises a first compression spacer located between said inlet and said three dimensional structure comprising graphene oxide and a second compression spacer located between said outlet and said three dimensional structure comprising graphene oxide.

Applicants disclose the modular solid nano-bioreactor according to the first eight paragraphs of this section titled “Modular Solid Nano-Bioreactor”, said modular solid nano-bioreactor comprising three or more compression spacers, at least one compression spacer being located between said inlet and said three dimensional structure comprising graphene oxide and at least one compression spacer located between said outlet and said three dimensional structure comprising graphene oxide and the remaining compression spacer being located within said three dimensional structure comprising graphene oxide.

Applicants disclose the modular solid nano-bioreactor according to the first eight paragraphs of this section titled “Modular Solid Nano-Bioreactor”, said modular solid nano-bioreactor comprising one or more spacers each said spacer is independently selected from the group consisting of a spring, a ring, or a compression spacer.,

Applicants disclose the modular solid nano-bioreactor according to the first eight paragraphs of this section titled “Modular Solid Nano-Bioreactor”, said modular solid nano-bioreactor, comprising one or more compression spacers each said spacer comprising: an exterior surface and an interior surface said interior surface defining an interior void comprising a top surface area and a bottom surface area; a plurality of stems protruding from said interior surface, said stems covering a portion of said void's top surface area and a bottom surface area, preferably said stems covering from about 15% to about 50% of said void's top surface area, more preferably said stems covering from about 15% to about 25% of said void's top surface area and bottom surface area and said stems protruding from about 60% to about 100% to the center of said void, preferably said stems protruding from about 60% to about 85% to the center of said void, more preferably said stems protruding from about 65% to about 80% to the center of said void, most preferably said stems protruding from about 72% to about 78% to the center of said void.

Applicants disclose the modular solid nano-bioreactor according to the previous paragraph wherein each said compression spacer comprises a first group of stems and a second group of stems, said first group of stems being longer than said second group of stems, each group of stems comprising at least three stems; or wherein each said compression spacer comprises at least six stems said stems having equal lengths or essentially equal lengths.

Applicants disclose the filter compression spacer of the previous two paragraphs, wherein said stems are branched.

### Process of Producing a Fermentation Product

A process of producing a fermentation product, said process comprising introducing one or more liquid nutrients into a modular solid nano-bioreactor according to any of claims 1 through 18, permitting said one or more liquid nutrients to be retained in said modular solid nano-bioreactor for a retention time of about one minute to twenty-four hours to produce a fermentation product and by product mixture, removing said fermentation product from said modular solid nano-bioreactor after said retention time and optionally separating said a fermentation product from said fermentation product and by product mixture, in one aspect said separation comprises centrifugation, filtration, and/or solvent extraction.

### Test Methods

The biofilm growth and cell density in the bioreactor for effective biofermentation of product is characterized via Microbial Enumeration Test (MET) following either ASTM D6974-20: Standard Practice for Enumeration of Viable Bacteria and Fungi in Liquid Fuels—Filtration and Culture procedure, or USP-NF M98800_01_01: Microbiological Examination of Nonsterile Products: Microbial Enumeration Test.

The modular solid bioreactors should provide a cell concentration per volume (cm³ for solid/gel and 1 mL for liquids) of 1×10⁴ cells to 1×10²⁰ cells.

A seeded 2.5 gallon modular solid bioreactor will produce product at a rate surpassing at a minimum 1 nL or 1 ng per day. Modular Solid bioreactors are expected to provide 15% higher fermentation performance than batch reactors at similar scales and growth conditions.

### EXAMPLES

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

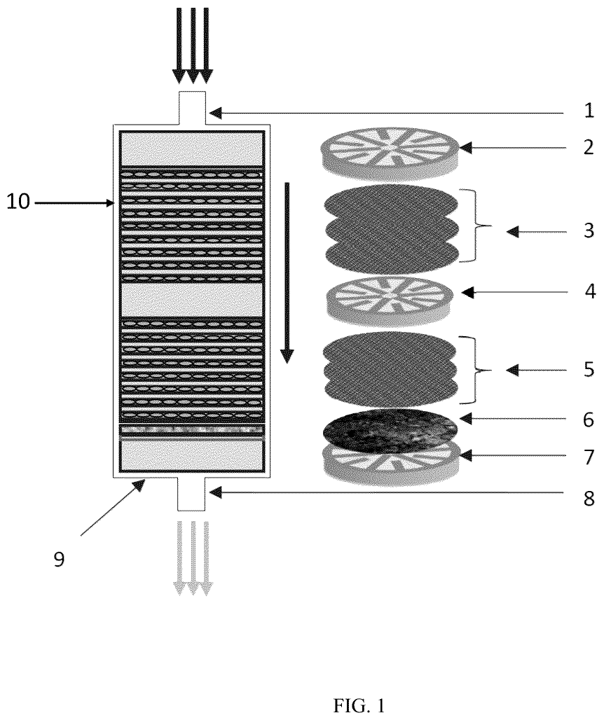

Example 1: Making and using the graphene oxide modular solid bioreactor. In order to accommodate the increasing flow rate required for multiple bioreactor systems and an acceptable pressure drop (<7 psi), the modular solid nano-bioreactor were designed on a modular cartridge design using graphene oxide coated metal meshes (graphene oxide coated scaffold) instead of 100% graphene oxide flakes. In addition to improved physical properties of the modular solid nano-bioreactor, the use of graphene oxide coated scaffold reduced the graphene oxide flake amount needed as the microbial cell growth medium. This was further enhanced by adding a graphene oxide flake bed at the inlet and outlet region of the bioreactor cartridge. The use of the graphene oxide scaffold (graphene oxide mesh) is considered to have increased the contact by providing multiple paths for microbial inoculum to fill the voids, allowing efficient cell growth and biofilm formation. Testing up to a 2 cm/s face velocity has shown that the graphene oxide coated scaffold is stable. Graphene oxide (GO) nanomaterial supports including coatings, solids, pack bed, solgels, flakes, 3D printed and liquid with enhanced microbial growth and modulation, biofilm formation, and enhanced biofermentation capacity are incorporated into a ruggedized modular solid bioreactors comprised of graphene oxide, graphene oxide scaffolds, spacer tools, which is all assembled into a modular solid nano-bioreactor. Between the graphene oxide scaffolds of the modular solid nano-bioreactor pack, graphene oxide microbial growth support in the form of flakes (packed bed), coated material, semi-solid or gel materials with or without cells embedded in the material, 3D printed materials, or graphene oxide liquid solution are integrated to fill the void spaces and promote cell growth and biofilm formation. Multiple modular solid nano-bioreactors can be added in a manifold configuration to increase the scale of biofermentation production in a modular fashion. Alternatively, the dimensions of the graphene oxide modular bioreactors can be increased to increase production capacity. The modular solid bioreactor systems is used by adding or embedding microorganisms or cells to the GO solid support, and then flowing at a low volume flow rate a nutrient media for growth that enters through the inlet port of the bioreactors and exits through the exit port of the modular bioreactors. The cells secrete products in the liquid effluent that it is final collected, concentrated and purified for use. The modular solid nano-bioreactor shown in FIG. 1 is made of 103 graphene oxide coated 3.84″ diameter 115 micron Dutch weaved wire cloth type mesh (the scaffold) made of 316 stainless steel loaded with ˜60 mg of graphene oxide on each mesh. A 2.9 g graphene oxide flake bed may be used at the bottom of the modular solid nano-bioreactor and is supported by a graphene oxide coated scaffold. Void spaces with the solid bioreactor are filled with cells by letting them grow to form biofilms or by filling the void with graphene oxide gel with embedded cells. Spacers are placed at the inlet above the graphene oxide coated scaffold stack and at the outlet below the mesh supporting the graphene oxide flake bed. The graphene oxide coated mesh stack with the graphene oxide flake bed is housed in a 4″ long, 3.84″ inside diameter housing with a spacer at the inlet and the outlet. The spacer is 0.49″ thick with 6 short (3 mm wide, 30 mm long) and 6 long stems (3 mm wide, 35 mm long) that extend into the center of the spacer and connected to a ring (4 mm wide) on the perimeter. The inlet and outlet to the modular solid nano-bioreactor is 1″ in diameter. The graphene oxide flakes used in the modular solid nano-bioreactor bed were cut to size from large graphene oxide flakes in a chopper and sieved to retain flakes >850 microns.

Example 2. Making and using the graphene oxide scaffold. Application of a graphene oxide coating onto a metal mesh to develop a graphene oxide scaffold for the modular solid nano-bioreactor is required a method that provided a good adhesion to the metal and be able to achieve the required mass loading to provide the required filtration efficiency. An approach that could support a commercial operation for coating the mesh that was independent of the mesh size was of interest. Different concentrations of the graphene oxide solution were applied to various mesh sizes used during the modular solid nano-bioreactor development showed that the surface tension in the graphene oxide solution be sufficient to attach to the mesh without forming a film that would flake and detach. Graphene oxide coating may be achieved by multiple processes including dipping, spraying, additive manufacturing, others. Testing showed that by adjusting the concentration of the graphene oxide solution according to the metal mesh size and drying at a low temperature to avoid altering the graphene oxide characteristics allows forming a graphene oxide coating that adheres to the metal mesh. Testing also showed that applications of multiple coatings by repeating the coating process allows increasing the graphene oxide mass loading on the metal mesh. Specifically, a concentration of 1 mg/ml of graphene oxide solution applied to 0.87″ and 3.84″ diameter metal mesh demonstrated that a similar mass loading could be achieved per unit area of mesh and that each coating added a similar amount of graphene oxide over several applications of the coating. Table 1 shows that the graphene oxide added to the metal mesh per unit area during each coating application is proportional to the area of the disks. In each coating application, the metal mesh was dipped in the graphene oxide solution and oven dried at a temperature of 50° C.

TABLE 1
Graphene Oxide Loading Proportional to Coated Scaffold Area

| Coating # | Av. 4″ | Av. 1″ | 4″:1″ Ratio |
|---|---|---|---|
| 1 | 7.37 | 0.39 | 19 |
| 2 | 13.55 | 0.61 | 22 |
| 3 | 21.77 | 0.96 | 23 |
| 4 | 27.60 | 1.30 | 21 |
| 5 | 37.32 | 1.86 | 20 |
| 6 | 44.37 | 2.24 | 20 |
| 7 | 49.70 | 2.42 | 21 |
| 8 | 57.58 | 2.82 | 20 |
| 9 | 60.63 | 3.03 | 20 |

Example 3. Making and using the graphene oxide hydrogels with and without embedded cells. Graphene oxide nanomaterials are combined with gelling agents to produce solid and semi-solid materials conducive to cell growth.

Example 4: Making and using graphene oxide. Graphene oxide samples were prepared using a Hummers method with modifications to improve the quality and purity of graphene oxide samples. In a typical graphene oxide synthesis using Hummers method, first the graphite powder was expanded and slightly oxidized using H₂SO₄, NH₄(S₂O₈) and P₂O₅. The product is further oxidized by manganese heptoxide (Mn₂O₇) as an oxidizer. Mn₂O₇ is formed by the reaction between potassium permanganate (KMNO₄) and sulfuric acid (H₂SO₄) in the temperatures below 10° C. Mn₂O₇ is a greenish viscous solution and it is formed due to the dehydration of two HMnO₄ molecules by H₂SO₄. Mn₂O₇ shows a highest reactivity between 35-55° C. and it reduces to MnO₂ violently above 55° C. Mn₂O₇ oxidizes the graphite surface to form oxygen functional groups, such as carboxylic, carbonyl, hydroxyl and ether. In the presence of water, these oxygen functional groups on the surface graphite sheets produce hydrogen bonds with water molecules and increase the inter layer distance between graphene layers to convert graphite oxide to graphene oxide. Scale-up preparation of graphene oxides to 8 grams. Concentrated H₂SO₄ (50 mL) in a 500 mL flask was heated to 80° C., to which (NH₄)₂S₂O₈ (4.5 g) and P₂O₅ (4.5 g) were added. The mixture was stirred until the reagents were completely dissolved. The graphite powder (5 g) was added, and the resulting mixture was heated at 80° C. for 4.5 h. Upon being cooled to room temperature, the reaction mixture was diluted with water (1250 mL) and kept for ˜12 h. It was then filtrated and washed repeatedly with water, followed by drying in a vacuum oven. The solid sample (5 g) was added to concentrated H₂SO₄ (200 mL) in a 500 mL flask cooled in an ice bath. The mixture was added slowly to KMnO₄ (15 g over 40 min), during which the temperature was kept at <10° C. The reaction mixture, with a change in color from black to greenish brown, was heated at 35° C. for 2 h, followed by dilution with water (400 mL—Caution: the temperature must be kept at <35° C. throughout) and further stirring for 2 h. The reaction mixture was poured into a large beaker, to which water (1250 mL) and then aqueous H₂O₂ (30%, 50 mL) were added. Bubbles from the aqueous mixture along with a color change to brilliant yellow were observed. After the mixture was allowed to settle for ˜12 h, the clear supernatant was decanted, and the sediment was washed repeatedly with aqueous H₂SO₄ (5 wt %)-H₂O₂ (0.5 wt %) and HCl solution (10 wt %), followed by washing repeatedly with water until no layer separation was observed after centrifugation. The sample was then dialyzed against water for 7 days to yield a clean aqueous dispersion of GOs. The water was removed using rotary evaporation and graphene oxide was recovered as a black flaky powder.

Example 7: Sizing the modular solid nano-bioreactor for larger production rates. The configuration used in the modular solid nano-bioreactor could be scaled up for larger flow rates using two methods. One method involves a modular configuration using a manifold that provides equal flow. When much larger flow rates are required, it will typically be necessary to scale up a modular solid nano-bioreactor. To achieve similar production efficiency, the following steps are typically required when scaling the modular solid nano-bioreactor:
a) apply a similar graphene oxide loading/unit area of the metal mesh (5 mg/square inch)
b) use a graphene oxide coated mesh stack that provides similar retention time by increasing the number of mesh and or the area of the mesh to provide an acceptable pressure loss.
c) use a similar graphene oxide flake mass/unit area of the metal mesh (0.25 g of graphene oxide flakes/square inch) in the graphene oxide flake bed at the outlet of the modular solid nano-bioreactor.
d) fill the void spaces with cells by letting them grow to form biofilms or fill the void spaces with graphene oxide gel with embedded cells.
e) use spacers at the inlet and outlet scaled according to the modular solid nano-bioreactor housing inside diameter and the inlet/outlet sizes that provides an acceptable pressure loss.

Every document cited herein, including any cross-referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and process, and illustrative examples shown and described. Accordingly, departures may be made from such details without departing from the scope of the general inventive concept.

What is claimed is:

1. A modular solid nano-bioreactor comprising walls that form a three dimensional housing, said housing comprising an interior chamber, at least one inlet passing through at least one of said walls to said interior chamber and at least one outlet passing through at least one of said walls to said interior chamber, said housing's chamber comprising a three dimensional structure comprising graphene oxide seeded with a microorganism and/or a cell, said microorganism and/or cell being embedded in and/or attached to the surface of said graphene oxide.

2. The modular solid nano-bioreactor according to claim 1 wherein said graphene oxide is selected from Graphene oxide flakes, graphene oxide powder, graphene oxide hydrogels and/or solvgels, graphene oxide liquid solution.

3. The modular solid nano-bioreactor according to claim 1 wherein:
a) said microorganism is selected from the group consisting of bacteria, fungi, protist, archaea and mixtures thereof; and/or
b) said cell is selected from the group consisting of eukaryotic cells, prokaryotic cells and mixtures thereof.)

4. The modular solid nano-bioreactor according to claim 3 wherein:
a) said bacteria said bacteria are selected from the group consisting of gram negative bacteria, gram positive bacteria and mixtures thereof;
b) said fungi are selected from the group consisting of filamentous fungi, yeasts and mixtures thereof;
c) said archaea are selected from the group consisting of methanobacterium formicicum, Methanobrevibacter smittii, Methanobrevibacter ruminantium, Halobacterium halobium, Halobacterium salinarum, Methanococcus voltae, and mixtures thereof;
d) said protist are selected from the group consisting of algae, amoeba, euglena and mixtures thereof; and
e) said eukaryotic cells are selected from the group consisting of mammalian cells, insect cells, plant cells and mixtures thereof.

5. The modular solid nano-bioreactor according to claim 4 wherein:
a) said gram negative bacteria are selected from the group consisting of Synechococcus, Synechocystis, Pseudomonas aeruginosa, Pseudomonas stutzeri, Pseudomonas frederiksbergensis, Pseudomonas putida, Pseudomonas, Acinetobacter venetianus, Acinetobacter calcoaceticus, Marinobacter hydrocarbonoclasticus, Marinobacter alkaliphilus, Marinobacter maritimus, Marinobacter squalenivorans, Rhodovulum imhoffii, Rhodovulum sulfidophilum, Achromobacter spanius, Achromobacter xylosoxidans, Achromobacter denitrificans, Desulfovibrio alaskensis, Desulfovibrio desulfuricans, Escherichia coli, Synechocystis sp., Synechococcus elongatus, and mixtures thereof, and said gram positive bacteria are selected from the group consisting of Gordonia sihwensis, Nocardioides luteus, Bacillus lincheniformis, Dietzia psychralcaliphila and mixtures thereof;
b) said filamentous fungi are selected from the group consisting of Hormoconis resinae, Fusarium fujikuroi, Byssochlamys nivea, Apergillus versicolor, Byssochlamys sp BYSS01, Lecanicillium sp Lec01, and mixtures thereof, and said yeasts are selected from the group consisting of Saccharomyces cerevisiae, Schizosaccharomyces pombe, Yarrowia lipolytica, Meyerozyma guilliermondii, Candida tropicalis, Candida albicans, Debaryomyces, Rhodoturula mucilaginosa, Rhodoturula toruloides and mixtures thereof;
c) said algae are selected from the group consisting of Clamydomonas, volvox, Spirogyra, chlorella and mixtures thereof; and
d) said mammalian cells are human cells, mouse cells, monkey cells and mixtures thereof.

6. The modular solid nano-bioreactor according to claim 1 wherein said three dimensional structure comprising graphene oxide is selected from the group consisting of:
a) a three dimensional structure comprising porous graphene oxide, said three dimensional structure comprising porous graphene oxide not comprising a support scaffold; and/or
b) a three dimensional structure comprising graphene oxide, said three dimensional structure comprising graphene oxide comprising a support scaffold comprising an exterior surface and optionally pores having walls said pores passing through said support scaffold, said graphene oxide being attached to at least a portion of said three dimensional structure comprising graphene oxide.

7. The modular solid nano-bioreactor according to claim 1 wherein said three dimensional structure comprising graphene oxide is selected from the group consisting of:
a) a three dimensional structure comprising a graphene oxide cartridge, graphene oxide gel and/or graphene oxide mesh; and/or
b) a three dimensional structure comprising graphene oxide, said three dimensional structure comprising graphene oxide comprising a support scaffold comprising an exterior surface and optionally pores having walls said pores passing through said support scaffold, said graphene oxide being attached to at least a portion of said three dimensional structure comprising graphene oxide, said support scaffold comprising pores having walls said pores passing through said support scaffold.

8. The modular solid nano-bioreactor according to claim 1 wherein said three dimensional structure comprising graphene oxide is selected from the group consisting of:
a) a said three dimensional structure comprising a graphene oxide cartridge, and/or graphene oxide mesh; and/or
b) a three dimensional structure comprising graphene oxide, said three dimensional structure comprising graphene oxide comprising a support scaffold comprising an exterior surface and optionally pores having walls said pores passing through said support scaffold, said graphene oxide being attached to at least a portion of said three dimensional structure comprising graphene oxide, said support scaffold comprising pores having walls said pores passing through said support scaffold, said graphene oxide being attached to the walls of said pores and said support scaffold's exterior surface.

9. The modular solid nano-bioreactor according to claim 8 wherein said support scaffold comprises a graphene oxide mesh, a bead, a fiber, a tube, a nanotube, and/or nanoparticle all said bead, a fiber, a tube, a nanotube, and nanoparticle having a graphene oxide attached thereto.

10. The modular solid nano-bioreactor according to claim 1 a compression spacer located between said inlet and said three dimensional structure comprising graphene oxide or between said outlet and said three dimensional structure comprising graphene oxide.

11. The modular solid nano-bioreactor according to claim 10 wherein said compression spacer is located between said inlet and said three dimensional structure comprising graphene oxide.

12. The modular solid nano-bioreactor according to claim 1 wherein said modular solid nano-bioreactor comprises a first compression spacer located between said inlet and said three dimensional structure comprising graphene oxide and a second compression spacer located between said outlet and said three dimensional structure comprising graphene oxide.

13. The modular solid nano-bioreactor according to claims 1, said modular solid nano-bioreactor comprising three or more compression spacers, at least one compression spacer being located between said inlet and said three dimensional structure comprising graphene oxide and at least one compression spacer located between said outlet and said three dimensional structure comprising graphene oxide and the remaining compression spacer being located within said three dimensional structure comprising graphene oxide.

14. The modular solid nano-bioreactor according to claim 1 comprising one or more spacers each said spacer is independently selected from the group consisting of a spring, a ring, or a compression spacer.

15. The modular solid nano-bioreactor according to claim 1 comprising one or more compression spacers each said spacer comprising:
a.) an exterior surface and an interior surface said interior surface defining an interior void comprising a top surface area and a bottom surface area;
b.) a plurality of stems protruding from said interior surface, said stems covering a portion of said void's top surface area and a bottom surface area.

16. The modular solid nano-bioreactor according to claim 15, wherein said stems cover from about 15% to about 50% of said void's top surface area.

17. The modular solid nano-bioreactor according to claim 15, wherein said stems cover from about 15% to about 25% of said void's top surface area and bottom surface area and said stems protrude from about 60% to about 100% to the center of said void.

18. The modular solid nano-bioreactor according to claim 17, wherein said stems protrude from about 60% to about 85% to the center of said void.

19. The modular solid nano-bioreactor according to claim 18, wherein said stems protrude from about 65% to about 80% to the center of said void.

20. The modular solid nano-bioreactor according to claim 19, wherein said stems protrude from about 72% to about 78% to the center of said void.

21. The modular solid nano-bioreactor according to claim 15, wherein each said compression spacer comprises a first group of stems and a second group of stems, said first group of stems being longer than said second group of stems, each group of stems comprising at least three stems.

22. The modular solid nano-bioreactor according to claim 15, wherein each said compression spacer comprises at least six stems said stems having equal lengths or essentially equal lengths.

23. The modular solid nano-bioreactor according to claim 15, wherein said stems are branched.

24. A process of producing a fermentation product, said process comprising introducing one or more liquid nutrients into a modular solid nano-bioreactor according to claim 1, permitting said one or more liquid nutrients to be retained in said modular solid nano-bioreactor for a retention time of about one minute to twenty-four hours to produce a fermentation product and by product mixture, removing said fermentation product from said modular solid nano-bioreactor after said retention time and optionally separating said a fermentation product from said fermentation product and by product mixture.

25. The process of claim 24 wherein said fermentation product from is separated from said fermentation product and by product mixture by a process comprising centrifugation, filtration, and/or solvent extraction.
